(19) United States
(12) Patent Application Publication
(10) Pub. No.: US 2025/0366763 A1
(43) Pub. Date: Dec. 4, 2025

(54) SYSTEM FOR CHARACTERIZING A HEART RHYTHM AND ASSOCIATED METHOD

(71) Applicant: SORIN CRM SAS, CLAMART (FR)

(72) Inventors: Pierre MAISON-BLANCHE, LEVALLOIS-PERRET (FR); Sylvain CHRISTOPHLE-BOULARD, SAINTE-GENEVIEVE-DES-BOIS (FR); Quentin FLEURY, NOZAY (FR); Rémi DUBOIS, MERIGNAC (FR)

(21) Appl. No.: 18/874,920

(22) PCT Filed: Oct. 11, 2023
(86) PCT No.: PCT/EP2023/078256

§ 371 Date: Dec. 13, 2024

(30) Foreign Application Priority Data
Oct. 11, 2022 (FR) FR2210423
Oct. 11, 2022 (FR) FR2210425

(51) Int. Cl. A61B 5/352; A61B 5/361; G16H 50/20

(52) U.S. Cl. CPC A61B 5/352; A61B 5/361; G16H 50/20

(57) ABSTRACT

A system for characterizing a heart rhythm configured to generate, by computer, for each arrhythmia of a set of arrhythmias, from descriptors of at least one lead of an electrocardiogram acquired during a time window, indicators of probability of presence of the arrhythmia over the time window, includes generating indicators of probability of presence of the arrhythmia in the time window using a classifier using values of the set of at least one lead, generating indicators of probability of presence of the arrhythmia in the time window, using a second classifier using first portions including a part preceding the R wave, of combinations of second portions of beats of the set of at least one lead, and generating indicators of probabilities of presence of the arrhythmia in the time window using a classifier using a set of statistical indicators representative of the R-R interval distribution.

### FIELD OF THE INVENTION

The field of the invention is that of the characterization of heart rhythms in the context of their detection on recordings of a biological signal. The detection of heart rhythm abnormalities allows a specialist to put in place a possible treatment either to relieve symptoms or to prevent more serious, potentially lethal, abnormalities.

The characterization of a patient's heart rhythm is performed by conventionally using an electrocardiogram (ECG). An ECG is the recording of the electrical activity of heart cells on the body surface or subcutaneously. An ECG comprises one or more views (or “leads”) of this electrical activity acquired simultaneously by respective cutaneous or subcutaneous electrodes.

Electrocardiograms conventionally comprise 12 leads acquired over a duration of 10 seconds.

The series of electric waves of a heart beat are called by international convention, in their temporal order, P, Q, R, S and T. The P waves correspond to the electrical activity of the atria, and the QRS waves to the electrical activity of the ventricles. By convention, the R-R interval refers to the time between two consecutive heart beats. In the present patent application, heart beat means a part of an electrical signal comprising the R wave, for example, centered on the R wave, the duration of which is the R-R interval.

The use of miniature and portable devices for continuous recording of electrocardiograms, such as cardiac holters, is increasing sharply, due notably to the aging of the population. In addition, these recordings cover increasingly longer durations of 24 hours to 1 month, because their efficiency is directly linked to the duration of the recording. The usual analysis, by dedicated software, then supervised by a specialist becomes illusory. For example, a 30-day recording comprises 3,000,000 heart beats. 1% error out of 3,000,000 beats obliges the specialist to review a large number of false positives and, even more critically, the specialist risks missing out on false negatives.

There is therefore an increasing need for optimal characterization of heart rhythms to help rhythmologists detect arrhythmias.

### PRIOR ART

Automatic heart rhythm characterization systems processing ECGs based on artificial intelligence or not are known.

From the article “An artificial intelligence-enabled ECG algorithm for the identification of patients with atrial fibrillation during sinus rhythm: a retrospective analysis of outcome prediction.” of Zachi I Attia et Al, The Lancet, Volume 394, ISSUE 10201, P861-867, 2019, is known a method for detecting a signature of atrial fibrillation from 12 leads of an ECG acquired simultaneously over a duration of 10 seconds. This method uses a convolutional neural network trained from ECG leads on which the presence or absence of an atrial fibrillation signature has been verified previously by specialists.

One drawback of this solution lies in the need to train the artificial neural network on a very large number of ECGs to obtain reliable and sensitive results.

One aim of the invention is to limit this drawback.

### SUMMARY OF THE INVENTION

To this end, the invention relates to a system for characterizing a heart rhythm comprising a data processing unit configured to generate, by computer, for each arrhythmia of a set of arrhythmias, from descriptors of a set of at least one lead of an electrocardiogram of an individual acquired during a time window, a set of indicators of probability of presence of the arrhythmia over the time window, the generation comprising:
generating a first set of indicators of probability of presence of the arrhythmia in the time window using a first classifier using electrical values of the set of at least one lead,
generating a second set of indicators of probability of presence of the arrhythmia in the time window, using a second classifier using first portions including a portion preceding the R wave, combinations of second portions of beats of the set of at least one lead,
generating a third set of indicators of probability of presence of the arrhythmia in the time window using a third classifier using a set of statistical indicators representative of the R-R interval distribution of the electrocardiogram.

Advantageously, the first portions are devoid of R wave.

According to one particular embodiment, the data processing unit is configured to generate a set of global indicators comprising global indicators of probabilities of presence of arrhythmias in the time window, using a global classifier using an input vector generated from the sets of indicators generated for the arrhythmias.

Advantageously, each of the first, second, and third classifiers is configured to discriminate a sinus rhythm from the arrhythmia.

Advantageously, each of the first, second, and third classifiers is a trained classifier.

According to one particular embodiment, the data processing unit is configured to generate the input vector from the sets of indicators generated for the arrhythmias, the input vector comprising, among the indicators of the sets of indicators generated for the arrhythmias, only each indicator of a selection of indicators, the system comprising a selector making it possible to select and/or deselect at least one of the indicators so that the selection of indicators is composed of each selected indicator.

Advantageously, the data processing unit is configured to define a sequence of states of the heart rhythm of the individual taken among the arrhythmias and the sinus rhythm, from sets of global indicators generated for successive time windows.

Advantageously, the definition of the sequence of states uses a Viterbi algorithm configured to determine the most probable sequence of states likely to be obtained by a hidden Markov model from the sets of global indicators.

Advantageously, the first classifier comprises a convolutional neural network and/or the third classifier comprises a convolutional neural network.

Advantageously, the arrhythmias are auricular fibrillation and auricular flutter.

Advantageously, the data processing unit comprises a data processing sub-unit configured to generate the set of statistical indicators representative of the time distribution of the R-R intervals of the set of at least one lead from a sequence of R-R intervals of the electrocardiogram, the generation comprising:
Parameterizing a Gaussian mixture model so as to obtain parameters of the Gaussian mixture comprising averages of Gaussians making up the model,
Generating first statistical indicators of the set of statistical indicators representative of the distribution of the R-R intervals of the electrocardiogram from the Gaussian averages,

And/or the following second set of steps:
Generating a sequence of relationships between consecutive R-R intervals,
Generating input indicators of probabilities of presence of the arrhythmia from the sequence of relationships using an R-R ratio classifier.

Advantageously, the system comprises an acquisition system comprising electrodes configured to acquire the set of at least one lead of the electrocardiogram of the individual.

Advantageously, the acquisition system is capable of forming an acquisition device comprising the data processing sub-unit.

Advantageously, the set of at least one lead comprises several leads.

In other words, the system according to the invention is configured to implement a method according to the invention.

The method according to the invention is a method for characterizing a heart rhythm comprising the generation, implemented by computer, for each arrhythmia of a set of arrhythmias, from descriptors of the set of at least one lead of an electrocardiogram of an individual acquired during the time window, of a set of indicators of probability of presence of the arrhythmia in the time window, the generation comprising:
generating a first set of indicators of probability of presence of the arrhythmia in the time window using a first classifier using values of the set of at least one lead,
generating a second set of indicators of probability of presence of the arrhythmia in the time window, using a second classifier from first portions, preceding the R wave, of combinations of second portions of beats of the set of at least one lead,
generating a third set of indicators of probability of presence of the arrhythmia in the time window using a third classifier using a set of statistical indicators representative of the R-R interval distribution of the electrocardiogram.

The invention also relates to a computer program product comprising a readable information medium, on which a computer program comprising program instructions is stored, the computer program being loadable on a data processing unit and adapted to drive the implementation of steps or of the steps of the method according to the invention, when the computer program is implemented on the data processing unit.

The invention also relates to a readable information medium including program instructions forming a computer program, the computer program being loadable on a data processing unit and adapted to drive the implementation of steps or of the steps of the method according to the invention when the computer program is implemented on the data processing unit.

### BRIEF DESCRIPTION OF THE FIGURES

Other features and advantages of the invention will become clearer upon reading the following detailed description, in reference to the appended figures, that illustrate:

FIG. 1: a schematic view of an exemplary system according to the invention,

FIG. 2: a schematic view of a lead,

FIG. 3: a view of functional blocks of an exemplary system according to the invention,

FIG. 4: a representation of a heart beat,

FIG. 5: a block diagram of the steps of an example of the method according to the invention.

### DESCRIPTION OF THE INVENTION

The invention relates to a method for characterizing a heart rhythm and a system SYS configured to implement the method according to the invention.

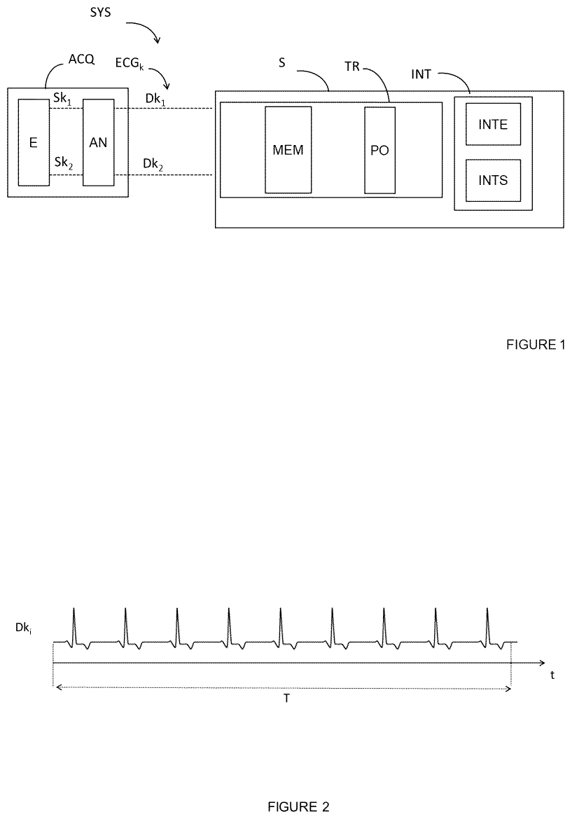

The system SYS comprises, for example, as seen in FIG. 1, an acquisition system ACQ of an electrocardiogram, noted ECGk in the following text, of a patient during an acquisition time window noted Tk in the following text.

### Acquisition System

The acquisition system ACQ comprises a set E of cutaneous or subcutaneous electrodes making it possible to acquire simultaneously during a same acquisition time window Fk of duration T, a set of at least one elementary analog signal Ski with i=1 to N and N is an integer greater than or equal to 1 representing the number of leads of the electrocardiogram ECGk acquired over the time window.

N is advantageously between 1 and 12, but may alternatively be greater than 12.

Each elementary signal Ski represents the evolution of the value Vᵢ (t) of the potential difference between electrodes of the set E as a function of the time t during the duration T of the acquisition window Fk.

The acquisition system ACQ also comprises an analog-to-digital converter AN to convert the elementary analog signals Ski into digital signals called leads Dkᵢ in the following text, the signals being sampled at a predetermined sampling frequency.

The sampling frequency is advantageously between 100 Hz and 2000 Hz, preferably between 100 Hz and 1000 Hz. It is, for example, equal to 200 Hz.

The acquisition device may comprise a combiner configured to combine digital signals such that at least one lead is a combination of digital signals.

Each lead Dkᵢ is one of the digital signals composing the electrocardiogram ECGk of the patient, acquired during the acquisition time window of duration T. It is a sequence of values of potential differences Vᵢ taken at regular time intervals over the duration T. An example of a lead Dkᵢ is schematically represented in FIG. 2.

The acquisition system may comprise lead processing modules, for example to filter the leads coming from the analog converter, before delivering the leads Dkᵢ.

The duration T of the acquisition window is advantageously greater than or equal to 10 s. It is, for example, between 10 s and 30 min. For example, it is equal to 60 seconds. It is defined so that each lead comprises a plurality of heart beats.

The set E of electrodes may comprise at least one electrode intended to be installed on the surface of the skin and/or at least one electrode intended to be installed subcutaneously.

Alternatively, the system SYS is devoid of the acquisition system and is intended to receive leads from an ECGk of a patient acquired during the acquisition time window Fk by an external acquisition system, or directly from descriptors of such leads as we will see in the following description.

### Processing System

The system SYS comprises a processing system S configured to process, by computer, the set of at least one lead Dkᵢ or the descriptors of the set of at least one lead Dkᵢ so as to generate indicators of probabilities of presence of arrhythmias of a predetermined set of arrhythmias, during the acquisition window Fk.

Probability of presence of an arrhythmia during the acquisition window Fk generated from a lead is understood to mean the probability that the arrhythmia is present in the time window Fk.

In the example in FIG. 2, the set of arrhythmias consists of auricular fibrillation, noted FA, also known as atrial fibrillation and auricular flutter, noted FLA, also known as atrial flutter.

This example is not limiting. In general, the set of arrhythmias comprises several arrhythmias taken from auricular fibrillation, auricular flutter, ventricular tachycardia, supraventricular tachycardia, at least one bradycardia, for example atrioventricular block.

The system S comprises a data processing unit TR, for example a processor assembly PO comprising one or more processors, and a memory assembly MEM comprising one or more memories in which are stored the functional bricks of the system realized in the form of software or codes executable by the processor assembly PO. The data processing unit is configured to implement the method for characterizing arrhythmias by executing the functional bricks stored on the memory assembly MEM, i.e. using the different functional bricks, and described below. The steps of the method for characterizing arrhythmias are the steps implemented by the execution of the different functional bricks.

### Functional Bricks

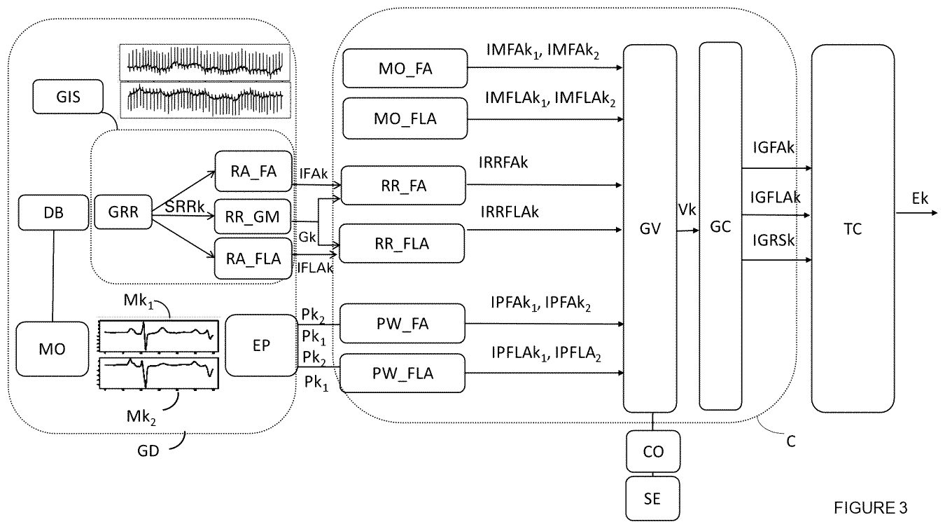

FIG. 3 shows an example of a processing system S on which functional bricks of the processing system S are shown.

The system S comprises a classifier C configured to generate a set of indicators of probability of presence of the arrhythmia in the time window Fk, for each time window Fk of a succession of time windows (with I=1 to K where K is the number of time windows) and for each arrhythmia of a set of arrhythmias FA, FLA. The set of indicators of probability of presence is generated from descriptors of the set of at least one lead Dkᵢ of an electrocardiogram ECGk acquired during the time window Fk.

The processing system S advantageously comprises a descriptor generator GD configured to generate the descriptors from the set of at least one lead Dkᵢ. Alternatively, the descriptor generator GD is external.

### Elementary Classifiers

To this end, the classifier C comprises a set of elementary classifiers for each arrhythmia. In other words, in the case of M arrhythmias, the classifier C comprises M sets of elementary classifiers. M is an integer greater than or equal to 2.

Thus, in the example of FIG. 3, the classifier C comprises a first set of elementary classifiers MO_FA, RR_FA, PW_FA associated with auricular fibrillation, i.e. configured to generate a first set of indicators IMFAK₁, IMFAK₂, IRRFAK, IPFAK₁ and IPFAK₂ representative of probabilities of presence of auricular fibrillation, and a second set of elementary classifiers MO_FLA, RR_FLA, PW_FLA associated with auricular flutter, i.e. configured to generate a second set of indicators IMFAK₁, IMFLAK₂, IRRFLAK, IPFLAK₁, IPFLAK₂ representative of probabilities of presence of auricular fibrillation FA.

The different elementary classifiers associated with a same arrhythmia, i.e. of a same set of elementary classifiers, belong to distinct groups in the sense that they are configured to generate the respective indicators from distinct respective descriptors of the different leads Dkᵢ or of the lead in the case of the use of a single lead. On the other hand, the elementary classifiers associated with different arrhythmias and using the same descriptors belong to a same group.

Advantageously, each elementary classifier is configured to discriminate a lead characterizing the presence of an arrhythmia from a lead characterizing the presence of a sinus rhythm, from the descriptors of a lead.

To this end, each classifier was previously trained from training lead descriptors, acquired over the same duration T and having the same sampling frequency as the leads of the set of at least one lead. The training leads comprise only first training leads characterizing a sinus rhythm and other training leads, the other training leads characterizing the presence of the arrhythmia treated by the classifier so as to discriminate a lead of an auricular fibrillation FA from a lead of a sinus rhythm RS.

Advantageously, each set of elementary classifiers associated with an arrhythmia comprises a first classifier, so-called morphological, a second classifier, so-called P wave analyzer, and a third classifier, so-called R-R analyzer.

These classifiers are each configured to distinguish one of the arrhythmias from the sinus rhythm based on one of the following analyzes, and (i) the morphology of the auricular electrical activity, (ii) the presence or absence of a P wave of auricular activity, (iii) the presence or absence of an irregular heart rhythm

In the patent application, the indicators delivered by the different elementary classifiers are noted XYk where X corresponds to the type of classifier and Y is the arrhythmia sought, i.e. whose classifier generates a probability of presence. k is the time order number of the time window Fk in the sequence of time windows.

In the example of FIG. 3, X=MO is noted for the morphological classifier, X=R-R for the R-R analyzer classifier and X=PW for the P wave analyzer classifier and Y=FA for auricular fibrillation and Y=FLA for flutter.

Some indicators are supplemented by an index i. The index indicators i are generated from characteristics of a first lead of index i, Dkᵢ, of the set of at least one lead. By way of example, the indicators IMFAK₁ and IMFLAK₁ are generated from characteristics of the first lead DK₁. The indicators IMFAK₂ and IMFLAK₂ are generated from characteristics of the second lead DK₂.

### Morphological Classifier

The so-called morphological classifier MO_FA, respectively MO_FLA, associated with a given arrhythmia FA, respectively FLA, is configured to process consecutive values Vᵢ of the lead(s) DK₁, Dk₂ to generate a first set of indicators IMFAK₁, IMFAK₂; respectively IMFLAK₁, IMFLAK₂, representative of probabilities of presence of the arrhythmia FA, respectively FLA, during the time window Fk.

Each indicator IMFAK₁, IMFAK₂, IMFLAK₁, IMFLAK₂ is generated from consecutive values Vᵢ of only one of the leads Dk₁, Dk₂. Each morphological classifier MO_FA, respectively MO_FLA, is configured to generate indicators IMFAK₁, IMFAK₂, respectively IMFLAK₁, IMFLAK₂ from the values of the lead(s) DK₁, Dk₂.

Advantageously, in the case of several leads, each morphological classifier MO_FA, MO_FLA is configured to process the consecutive values of the two leads Dk₁, Dk₂ so as to successively generate the indicators IMFAK₁ and IMFAK₂.

Advantageously, each morphological classifier MO_FA, MO_FLA is an artificial neural network, for example a convolutional neuron network or CNN. Alternatively, the classifier is a recurrent neural network or transformer.

Each morphological classifier MO_FA, respectively MO_FLA is advantageously an artificial neural network trained from consecutive values of leads acquired over a same duration and that of the acquisition window Fk with a same sampling frequency, some of which characterize a sinus rhythm and the others characterize the presence of the arrhythmia FA, respectively FLA, treated by the elementary classifier so as to discriminate a lead characterizing an auricular fibrillation FA from a lead characterizing a sinus rhythm RS.

This type of classifier and discriminant for arrhythmias resulting in distinct characteristic distributions from one arrhythmia to another and those of a patient with a sinus rhythm.

This is, for example, the case of auricular fibrillation and flutter. Indeed, auricular fibrillation is characterized by an irregular electrical activity whereas the electrical activity in the presence of a sinus rhythm is regular and is between 60 and 100 beats per minute whereas the electrical activity in the presence of an auricular fibrillation is between 300 and 600 cycles per minute.

Flutter is for its part characterized by a regular electrical activity. The heart rate in the presence of flutter is between 100 and 300 cycles per min.

### P Wave Analyzer Classifier

The classifier C further comprises a so-called P wave analyzer classifier for each arrhythmia.

Each P wave analyzer classifier PW_FA; respectively PW_FLA, associated with a given arrhythmia FA, respectively FLA, is configured to process combinations, for example, averages of predetermined portions of beats present on the respective lead(s) Dk₁, Dk₂, acquired during the acquisition window F to generate indicators IPFA₁, IPFA₂; respectively IPFLA₁, IPFLA₂, of probabilities of presence of the arrhythmia.

These portions are advantageously portions preceding the R wave and devoid of the R wave.

These portions are, for example, located only before the R wave.

These portions advantageously comprise the P depolarization wave temporally preceding the R wave in the case of sinus rhythm or, more generally, the part of the combination likely to comprise the P wave in the case of sinus rhythm. Advantageously, the combined, for example averaged, portions correspond to portions of the same predetermined duration preceding the R wave and devoid of the R wave.

Each portion is, for example; a portion of predetermined duration preceding the R wave. This duration is, for example, between 200 ms and 500 ms.

For example, the predetermined duration portion extends from a first instant to a second instant preceding the R wave and separated by a predetermined time difference from the previously detected R wave or the previously detected QRS complex.

Alternatively, each portion corresponds to a complete beat.

In another embodiment, the portion follows the R wave. It is, for example, located only after the R wave.

The system S advantageously comprises a combiner, for example an averager MO, of P waves configured to generate, for each lead Dk₁, respectively Dk₂, a combination, for example an average MK₁, Mk₂ of at least one subset of the beats present on the lead Dk₁, respectively Dk₂, and detected by the R wave detector. The combined beats, for example averaged, advantageously have the same duration equal to the beat period, are, for example centered on the R wave and have a predetermined duration, advantageously defined such that the beats used for the combination or averaging comprise the successive P, Q, R, S and T waves.

In other words, the combiner is configured to combine at least one subset of the beats of a lead so as to generate a signal that we call combination.

Alternatively, the R waves are subtracted from the combined waves before the combination or the R waves are subtracted from the combinations. This enables the classification to be facilitated.

The fact of making the combinations or averages of portions of the beats including only the P wave in the case of a sinus rhythm, or, more generally, the portion of the beat likely to comprise the P wave, makes it possible to facilitate the classification of the auricular activity.

The processing system S advantageously, but not necessarily, comprise a portion extractor EP configured to extract a portion PK₁, Pk₂ of one of the averages or combinations MK₁, Mk₂ of the beat, obtained from one of the leads DK₁, DK₂. This portion is, for example, the considered combination or average, it may comprise a portion preceding the R wave and the R wave, for example the entire combined or averaged PQRS complex, or instead a portion, for example of predetermined duration, of the combined or averaged beat, preceding the R wave and devoid of the R wave, i.e. including only part of the combination preceding the R wave. The suppression of the QRS complex of significantly higher intensity than that of the preceding part of the P wave, in particular in the case of auricular fibrillation or flutter, makes it possible to improve the performance of the classifier.

This portion is, for example; a portion of predetermined duration preceding the R wave. This duration is, for example, between 200 ms and 500 ms.

For example, the predetermined duration portion extends from a first instant to a second instant preceding the R wave and separated by a predetermined time difference from the previously detected R wave or the previously detected QRS complex.

Each P wave analyzer classifier PW_FA, PW_FLA is configured to generate indicators IPFAK₁, IPFAK₂; respectively IPFLAK₁, IPFLAK₂ of probabilities of presence of the arrhythmia given from portions Pk₁, Pk₂ of combinations or averages of beats of the leads Dk₁, DK₂.

Each P wave analyzer classifier PW_FA, PW_FLA is advantageously configured to generate each indicator of probability of presence of the arrhythmia IPFAK₁, IPFAK₂; IPFLAK₁, IPFLAK₂ from one of the portions Pk₁, Pk₂.

In other words, each P wave analyzer classifier PW_FA, PW_FLA is advantageously configured to generate each indicator of probability of presence of the arrhythmia IPFAK₁, IPFAK₂; IPFLAK₁, IPFLAK₂ that it generates from a portion Pk₁, Pk₂ of a single combination generated from a lead.

Each P wave analyzer classifier, PW_FA, respectively PW_FLA is able to generate indicators IPFAK₁, IPFAK₂; respectively IPFLAK₁, IPFLAK₂ from the portions of the combinations or averages derived from the different lead(s) Dk₁, DK₂.

In other words, each P wave analyzer classifier PW_FA, PW_FLA is configured to generate different indicators of probability of presence of the arrhythmia IPFAK₁, IPFAK₂; IPFLAK₁, IPFLAK₂ from portions Pk₁, PK₂ generated from respective leads.

In the case of a single lead, each indicator generated by a P wave analyzer classifier is generated from a portion of a combination obtained from the lead.

Advantageously, in the case of several leads, each P wave analyzer classifier PW_FA, PW_FLA is configured to successively process the portions of the combinations derived from the different leads Dk 1, Dk₂, i.e. to process the leads consecutively, so as to successively generate the indicators IMFAK₁ and IMFAK₂.

Advantageously, each P wave analyzer classifier PW_FA, PW_FLA is an artificial neural network, for example a convolutional neural network.

Each P wave analyzer classifier PW_FA, PW_FLA is advantageously trained previously from portions of combinations, for example averages, of beats of training leads acquired over a same duration as the acquisition window Fk and some of which characterize the sinus rhythm and the others the arrhythmia associated with the classifier so that the classifier is able to discriminate a characteristic portion of a sinus rhythm from a characteristic portion of the arrhythmia.

P wave analyzer classifiers are particularly interesting and discriminating for aiding in the detection of arrhythmias resulting in characteristic P wave shapes distinct from one arrhythmia to another and the characteristic P wave shape of a healthy patient.

This is, for example, the case of auricular fibrillation and auricular flutter. Indeed, auricular fibrillation is characterized by the absence of a domed sinus P wave replaced by irregular f waves, whereas auricular flutter is characterized by a P wave in “saw teeth”, identical and regular, diphasic (the negative phase of which predominates: this is referred to as an F wave) whereas a sinus rhythm has a P wave.

Furthermore, a classifier is more accurate and therefore sensitive and reliable than using a threshold to discriminate a sinus rhythm from auricular fibrillation or flutter which are characterized by replacing a P wave with irregular or regular waves.

Alternatively, the combination is, for example, a beat median.

Alternatively and/or in addition, at least one set of elementary classifiers dedicated to an arrhythmia may comprise at least one other classifier, for example, a classifier receiving a combination or average of QRS complexes and a combination or average of P waves separately to discriminate for example an atrioventricular block from the sinus rhythm, with a number of P waves greater than the number of QRS, or a sequence of P waves not followed by QRS.

It is possible alternatively and/or additionally to provide a classifier configured to discriminate characteristic leads of an arrhythmia from leads characteristic of the sinus rhythm from a Fourier transform of at least one of the aforementioned descriptors.

### R-R Analyzer Classifier or Regularity Classifier

The classifier C advantageously comprises an R-R interval analyzer classifier for each arrhythmia.

Each R-R interval analyzer classifier, RR_FA; respectively RR_FLA, associated with a given arrhythmia FA, respectively FLA, is configured to process statistical indicators representative of the distribution of the R-R intervals of the ECGk acquired during the acquisition window Fk to generate indicators IRRFAK; respectively IRRFLAK, representative of probabilities of presence of the arrhythmia FA, respectively FLA during the window Fk.

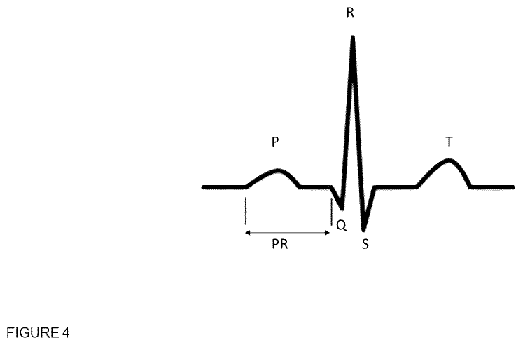

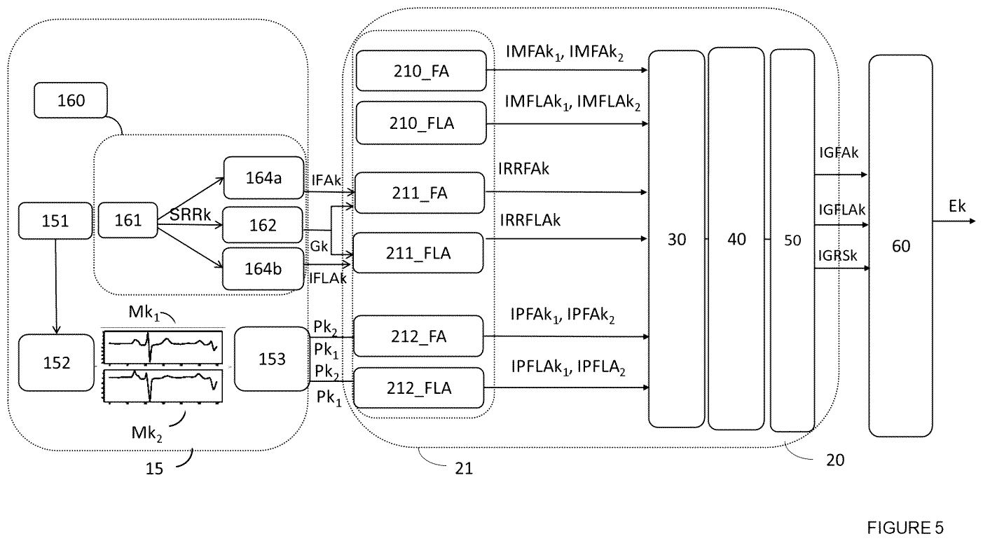

The part of a lead Dkᵢ corresponding to a beat consists of a succession in time of electric waves, the bearing of which is represented in FIG. 4. The P wave is the first, in time, and corresponds to the depolarization of the atria, has a low amplitude, and a dome shape in the case of sinus rhythm. Then, the PR interval translates the atrioventricular conduction time. The QRS complex reflects ventricular contraction, and finally the T wave ventricular repolarization following the QRS complex. By convention, the R peak is considered as a marker of ventricular systole. Since the R wave is most often the thinnest and the most ample part of the QRS complex, it is generally used by convention to localize the heart beat temporally with very good accuracy, in practice in the order of a thousandth of a second. Thus, the time interval between two successive R waves, also called by convention R-R interval, precisely characterizes the time separating two successive heart beats. The R-R interval is the instantaneous period of the ECG signal. The instantaneous heart rate is the inverse of the R-R interval.

The R-R interval is common to all the leads Dkᵢ of an electrocardiogram.

The descriptor generator GD advantageously comprises a statistical indicator generator GIS characterizing the distribution of the patient's R-R intervals during the acquisition window Fk from at least one lead of the set of at least one lead Dkᵢ. The statistical indicators generated are provided to the interval analyzer generators R-R_FA, RR_FLA as we will see in the following text.

The descriptor generator GD comprises a beat detector DB configured to detect and timestamp, i.e. to determine the times of occurrence or TOC, of consecutive R waves of at least one of the leads, the distribution of the R waves being the same for all the leads and optionally to detect and timestamp other waves, for example, P and/or Q and/or S and/or T from the lead.

The statistical indicator generator GIS comprises an RR series generator GR-R, SRRk, configured to generate an RR series, SRRk for the time window Fk, from at least one lead of the set of leads, for example the lead DKᵢ.

The R-R series generator GR-R comprises an R-R series builder configured to build the R-R series by calculating the R-R intervals between consecutive beats, from the time stamps of the consecutive R waves.

The series comprises H terms R-Rk(h) with h=1 to H-1, where H is the number of heart beats present in the lead Dkᵢ, h corresponds to the time order number of the first beat from which the R-R interval, considered in the lead Dkᵢ, is calculated.

The RR series, SRRk comprises successive values which are consecutive values R-Rk (h) of the instantaneous R-R intervals of the chosen lead Dkᵢ during the time window Fk, or which are determined from several leads.

Alternatively, the beat detector DB is configured to detect and timestamp one of the other depolarization waves (P, Q, S or T) in the consecutive beats. The RR series builder then uses these timestamps to build the RR series. However, the accuracy of the characterization of the time interval between two successive heart beats obtained by this method is less good than using R waves.

The statistical indicator generator GIS comprises an elementary statistical indicator generator RR_GM configured to generate, from the RR series, at least one statistical indicator Gk characterizing the statistical distribution of the intervals R-Rk (h) of the RR series, SRRk.

This elementary generator RR_GM is advantageously configured to generate, from the RR series, parameters of a Gaussian mixture model characterizing the distribution of the R-R intervals of the RR series, SRRk. In other words, the elementary generator RR_GM is configured to parameterize a Gaussian mixture model such that the Gaussian mixture model models the distribution of the R-Rs of the R-R series.

The Gaussian mixture model is commonly designated by the acronym GMM. The Gaussian mixture model is used to parametrically estimate the R-R distribution of the R-R series by modeling it as the sum of several Gaussians. The parameterization of the model consists in determining the variance, mean and amplitude of each of the Gaussians of the model. These parameter values are optimized according to the maximum likelihood criterion in order to get as close as possible to the distribution sought. This optimization is, for example, carried out using a known iterative procedure called maximization-expectation EM.

The elementary generator RR_GM is configured to calculate at least one elementary statistical indicator from at least one parameter of the parameter Gaussian mixture model. It is, for example, configured to calculate the ratio of the averages of the Gaussians of the model.

The Gaussian model comprises for example 1 or 2 Gaussians.

The statistical indicators derived from the parameters of the Gaussian model are characteristic and discriminant. In the case of a model comprising 3 Gaussians, the sinus rhythm comprises three Gaussians of same center due to the stability of the R-R interval. The ratio of the averages of the Gaussians is equal to 1. It is known that the heart cycle of a flutter can only take multiple values of a basic duration. The ratio of the averages of the Gaussians can therefore only take a predetermined number of values. This indicator is therefore particularly relevant to highlight the presence of auricular flutter. The distribution of the R-Rs is therefore modeled by respective Gaussians centered on these three respective values. The distribution of R-Rs in the case of auricular fibrillation is very irregular. The ratio of the Gaussian averages can therefore take an unlimited number of values.

In the non-limiting example of FIG. 3, the statistical indicator generator GIS comprises a generator of a sequence of relationships between consecutive R-R intervals given by the following formula:

$$RAk ( h ) = R - Rk ( h + 1 ) / R - Rk ( h ) for h = 1 at H - 2.$$

This series of relationships or ratios is characteristic of auricular fibrillation and flutter and makes it possible to distinguish these arrhythmias from each other and from a sinus rhythm.

Indeed, in the case of sinus rhythm, the ratios RA (h) all have a value substantially equal to 1 insofar as the heart rhythm is substantially stable and even if it increases, for example in the event of exertion, the R-R interval between a first beat and the second beat is substantially equal to the R-R interval between the second and third beat. In the case of flutter, the ratio of the RAk(h) can only take a certain number of known discrete values. In the case of auricular fibrillation, the ratios RA(h) can take on a large number of values.

Advantageously, the statistical indicator generator GIS comprises a generator of indicators of probabilities of presence of arrhythmias from the sequence of relationships between R-R intervals.

Advantageously, the generator comprises RR ratio classifiers, RA_FA, RA_FLA. Each ratio classifier is configured to discriminate one of the arrhythmias FA or FLA from the sinus rhythm.

To this end, each of the ratio classifiers is previously trained by series of ratios RAK (h) generated from training leads, some of which are characteristic of the arrhythmia considered and the others of the sinus rhythm, to discriminate the series of ratios derived from the sinus rhythm from those derived from the arrhythmia considered.

Each ratio classifier is configured to generate a statistical indicator IFAk, respectively IFLAK, which is an indicator of probability of presence of one of the arrhythmias FA or FLA considered from the series of ratios RAK (h).

Statistical indicators Gk, IFAk, IFLAK of the probability of presence of the respective arrhythmias in the window considered Fk are thus obtained.

The ratio classifiers are, for example, artificial neural networks. These classifiers are, for example, CNNs. Alternatively, at least one ratio classifier is a recurrent neural network or transformer.

The statistical indicators generated by the statistical indicator generator GIS are provided to the R-R analyzer classifiers, RR_FA and RR_FLA.

Advantageously, each R-R analyzer classifier, RR_FA, RR_FLA, is an artificial neural network, for example a multilayer perceptron, also known by the acronym MLP.

This classifier RR_FA, RR_FLA receives as input a vector composed of at least one statistical indicator generated by the generator GIS, for example the indicator Gk characterizing the distribution of the R-R intervals and the probability indicator generated for the arrhythmia considered IFAk, IFLAK.

Each R-R analyzer classifier RR_FA, RR_FLA is advantageously previously trained from the same vector generated by the statistical indicator generator GIS from leads, for example, measured during the same duration as the training window and of which the first characterize the arrhythmia considered and of which the others characterize the sinus rhythm, to discriminate these arrhythmias.

In the embodiment of FIG. 1, the acquisition system ACQ is an acquisition device, i.e. an object, connected to the system S, by communication means of the system SYS, for example wireless or wired allowing unidirectional or bidirectional communication between the system S and the acquisition device ACQ so that the acquisition device ACQ is able to transmit the leads to the system S. These are for example the communication means described in the following text.

In one particular embodiment, the processing unit comprises a first processing sub-unit configured to implement the descriptor generator GD from the set of at least one lead Dkᵢ or at least the statistical indicator generator GIS and, preferably the beat detector, and a second processing sub-unit configured to implement the classifiers C.

Advantageously, the acquisition system ACQ is capable of forming an acquisition device comprising the electrode assembly E and the first processing sub-unit. It comprises for example the electrode assembly and a housing capable of being mechanically connected to the electrode assembly or mechanically connected to this assembly and containing the first processing unit.

Advantageously, the housing further contains the analog-to-digital converter AN.

The acquisition device ACQ is then connected to the second processing sub-unit, by communication means of the system SYS, for example wireless or wired allowing unidirectional or bidirectional communication between the second processing sub-unit and the acquisition device ACQ such that the acquisition device ACQ is capable of transmitting the descriptors it has generated to the second processing sub-unit so that it can execute the classifier C.

Physically, each processing sub-unit may be implemented in one of the forms described above for the processing unit.

The acquisition device ACQ is advantageously portable or implantable on an individual subcutaneously.

### Segmentation of the Approach

The use of classifiers analyzing distinct descriptors of one or more leads in parallel to generate indicators of probability of presence of different arrhythmias requires less training data than a single classifier analyzing consecutive values of the lead(s) to obtain indicators of equivalent sensitivity or reliability. Indeed, the segmented analysis of the descriptors of the leads makes it possible to limit the number of degrees of freedom of the neural networks. The learning of the neural networks is facilitated.

Furthermore, the segmented approach is more robust to artifacts present on the leads.

Further, the fact of carrying out in parallel a morphological analysis of the signal, a statistical analysis of the temporal distribution of the ECG signal as well as an analysis of the P wave to generate indicators and generate global probability indicators from these probabilities is particularly efficient for discriminating an auricular flutter, an auricular fibrillation and a sinus rhythm. As seen previously, the descriptors analyzed by these different classifiers (morphology, P wave, statistical indicators of the distribution of R-R intervals) present specificities (shapes, values) typical of the different arrhythmias and of a sinus rhythm and discriminating between the different arrhythmias and the sinus rhythm and these descriptors are understandable for the end user because they simulate the basic rules of clinical electrocardiology.

In addition, the fact of performing different analyzes allows good performance to be maintained when one of the classifiers is less efficient for a lead with a particular aspect, for example in the event of an abnormally regular rhythm in the case of an auricular fibrillation.

The generation of different probability indicators for the same arrhythmia and the same lead, by the different classifiers, allows a rhythmologist to whom these indicators are provided, to improve his knowledge of the descriptors of the rhythm analyzed compared to the generation of a single indicator, which allows him to more easily identify an arrhythmia characterized by the ECGk or to identify an elementary classifier not relevant for the detection of an arrhythmia.

Furthermore, the fact of analyzing arrhythmias using different sets of elementary classifiers makes it possible to improve the performance of the classification and to limit the number of training data.

Further, the fact of analyzing the leads independently allows the rhythmologist to select the indicators he desires to deduce therefrom information on the rhythm, notably when a lead is manifestly degraded, for example when it has been improperly placed on the patient, allowing him to more easily and reliably identify an arrhythmia characterized by the ECGk.

The fact of generating time window probability indicators per time window makes it possible to provide information to the rhythmologist on a regular basis throughout the acquisition of a global ECG comprising a succession of time windows. This allows for faster detection of arrhythmias and more precise identification of the time of occurrence of arrhythmias.

### Input Vector Generator

The system advantageously comprises a vector generator VG configured to generate an input vector V from the sets of probability indicators generated by the classifier C.

The probability indicators are the probability indicators generated by the elementary classifiers of the classifier C.

The vector V is composed of a number A of components, where A is greater than or equal to the number L of probability indicators generated by the classifier C, i.e. generated by the elementary classifiers of the classifier C.

The vector Vk is capable of being such that each of a number L of its components is one of the probability indicators. Each probability indicator corresponds to a predetermined component, i.e. a predetermined order number in the order of the vector components.

In the non-limiting example of FIG. 3, the number L of indicators of probability of presence is equal to 10. 10 components of the vector V are reserved for the probability indicators, in the sense that their values are likely to be those of the L probability indicators.

When A is greater than L, the A-L other vector components may comprise values of other parameters such as age, patient history, and/or risk factors.

### Global Classifier

The classification system SC comprises a global classifier GC configured to generate a set of global indicators IGFAK, IGFLAK, IGRSk of probabilities of presence of arrhythmias FA, FLA, and sinus rhythm noted RS, over the time window Fk, from an input vector noted Vk.

The global classifier GC is previously trained from training vectors generated from training lead descriptors, by the classifier C, to discriminate vectors V coming from the different arrhythmias among themselves and with the sinus rhythm.

The test leads comprise test leads characteristic of the sinus rhythm, and sets of test leads characteristic of the respective arrhythmias, each set of test leads being characteristic of one of the arrhythmias.

The global classifier GC is, for example, an artificial neural network.

The neural network is for example a multilayer perceptron, a feedforward neural network. Alternatively, the global classifier is a forest of decision trees.

The generation of reliable global indicators from indicators generated by the different classifiers requires less training data than using a single classifier. This solution also offers improved sensitivity and greater robustness.

### Sequence of Leads

Advantageously, the acquisition system ACQ is configured to generate a sequence of sets of at least one lead, i.e. of ECGk with k=1 to K, acquired during K successive, for example consecutive, time windows F of a global acquisition window FG of global duration TG.

The global duration TG of the global acquisition window FG is advantageously between 20s and 30 days.

Advantageously, the successive time windows have the same duration.

The classifier C is configured to generate sequences of K sets of global indicators IGFAK, IGFLAK, IGRSk from the descriptors of successive sets of at least one lead DK₁, Dk₂. Each set of global indicators IGFAk, IGFLAK, IGRSk is generated from a single set of at least one lead DK₁, Dk₂ of the time window sequence Fk. The temporal order of the sets of global indicators IGFAK, IGFLAK, IGRSk is the same as that of the sets of leads Dk₁, Dk₂ from which they are respectively generated.

### Time Analyzer

Advantageously, the system S comprises a time analyzer TC configured to define a sequence of states Ek of the heart rhythm corresponding to the states Ek of the heart rhythm over the successive time windows Fk from the sequence of sets of global indicators IGFAK, IGFLAK, IGRSk generated for the successive time windows Fk.

Each state Ek is taken from the arrhythmias and the sinusoidal rhythm FA, FLA or RS.

The idea is to determine, from a sequence of K sets of global indicators IGFAK, IGFLAK, IGRSk, a sequence of states Ek of the heart rhythm in the successive time windows Fk.

The time analyzer TC is configured to generate the states Ek of the respective time windows Fk from the global indicators generated for other time windows, for example of all the other time windows. The time analyzer TC makes it possible to obtain a sequence of elementary states coherent with each other and with the known probabilities, for example learned, of switching from one state to the other from one time window to the other. It is known, for example, that when a rhythm is fluttering over a time window, the probability of remaining fluttering over the following time windows is very high. On the other hand, when the rhythm is an auricular fibrillation, the patient may switch to flutter, but with a low probability.

The time analyzer CT uses, for example, a model such as a hidden Markov model, also known by the acronym HMM, by implementing a Viterbi algorithm to generate the most probable state sequence from the sequence of global indicators that can be generated by the hidden Markov model.

The hidden Markov model used is parameterized.

In order to parameterize the hidden Markov model, the latter was previously subjected to a first training phase during which the hidden Markov model was trained from sequences of sets of global indicators IGFAk, IGFLAK, IGRSk labeled by a sequence of known states so as to generate a matrix of probabilities of transition between the possible states.

The hidden Markov model was also trained, in a second training phase, to learn, for each possible state (FA, FLA and RS), which observation (i.e. which set of global indicators IGFAK, IGFLAK, IGRSk) it corresponds to. This learning phase is performed from global training indicators generated, by the global classifier, from descriptors of one or more leads characteristic of the given state. The second training makes it possible to obtain an observation matrix. The separation of these two learning trainings makes it easy to learn.

The probability of starting in each state is, for example, predetermined. For example, it can be decided that the states are equally probable or that the probabilities of starting in the respective states are the same. Alternatively, these probabilities are calculated from a set of sequences of leads of which the sequences of states and therefore the first states are known.

Alternatively, the time analyzer TC comprises, for example, a self-attentive model or transformer. Alternatively, the time analyzer CT comprises a recurrent neural network, also known by the acronym RNN, for example, a recurrent network with “Long short-term memory”, also known by the acronym LSTM.

### Selector

Generally, the processing unit is configured to generate, for each arrhythmia of a plurality of arrhythmias FA, FLA, using a set of elementary classifiers, a set of indicators of probability of presence of the arrhythmia FA, FLA over a time window Fk using descriptors of a set of at least one lead of an electrocardiogram ECGk of a patient acquired over the time window Fk. The set of elementary classifiers comprises, for example, at least one of the elementary classifiers of FIG. 2 and, optionally, at least one other elementary classifier of the system S.

It is important to note that the indicators generated by the elementary classifiers are not always relevant, as their consideration by the global classifier leads to a deterioration in the reliability of the global indicators.

This is the case, for example, when an auricular fibrillation is associated with an electrical influx conduction disorder that modulates the R-R interval. Analysis of the R-R interval is not relevant to characterize an auricular fibrillation in this case, as consideration of the indicators generated by the R-R analyzer classifier degrades the reliability of the indicators generated by the global classifier. The reliability of the indicators generated by the morphological classifier is also degraded by the regularity of the R-R intervals. In this case, only the P wave analyzer classifier and the morphological classifier are relevant to generate the indicators.

Similarly, when a lead does not allow reliable indicators to be obtained, for example because it is noisy or has artifacts, for example due to muscle movement, the indicators generated from this lead, in particular by the P wave analyzer classifier from combinations of parts of this lead will not be reliable.

In order to improve the quality of the global indicators generated by the global classifier, the system advantageously comprises a selector INT, SE making it possible to select and/or deselect at least one of the indicators generated by the elementary classifiers so that only a selection of these indicators, i.e. only one selection taken from among these indicators, is taken into account by the global classifier GC, to generate the global indicators IGFAK, IGFLAK, IGRSk relating to the time window Fk considered.

In other words, only each indicator of the selection is taken into account by the global classifier GC to generate the global indicators IGFAk, IGFLAK, IGRSk relative to the time window Fk considered.

To this end, at least one of the indicators, for example each indicator, is capable of being alternately in a selected state and in a deselected state.

The selection of indicators comprises each indicator in the selected state. It may not comprise any indicator when all the indicators are in the deselected state or it may comprise one or more selected indicators.

The generator GV of the input vector V is configured to generate an input vector V comprising; among the indicators generated by the elementary classifiers for the arrhythmias, only each indicator of the selection of indicators.

Advantageously, the vector generator VG uses, among the indicators of probability of presence generated or likely to be generated for the time window by the sets of elementary classifiers dedicated to the different arrhythmias, only each indicator of probability of presence of the selection, to generate the input vector V.

The value of the other components of the input vector likely to take the values of the other indicators, i.e. reserved for the other indicators, is advantageously set at the same predetermined value. This value is, for example, equal to ½ or is representative of a probability of 1/2.

For example, in the case of 10 indicators of probability of presence, if the selection of indicators comprises two indicators, the values of the other 8 components of the vector reserved for the indicators of probability of presence are set at the same predetermined value, for example 1/2.

This makes it possible to limit the impact of non-selected indicators on the quality of the global indicators.

In a first embodiment described above, the data processing unit is configured, regardless of the number of indicators of the selection of indicators, to generate, for each arrhythmia of the plurality of arrhythmias, using the set of elementary classifiers, the set of indicators of probabilities of presence of the arrhythmia over the time window, using the descriptors of the set of at least one lead of the patient electrocardiogram acquired over the time window.

The processing unit is also configured to generate an input vector Vk whose components comprise, among the indicators of the sets of indicators of probability of presence generated for the arrhythmias over the time window, only each indicator of the selection.

The sets of indicators of probability of presence generated by the processing unit for the arrhythmias in the time window comprise each indicator of the selection of indicators.

If the selection is incomplete, in the sense that it does not comprise all the indicators of probability of presence likely to be generated by the elementary classifiers for a time window, the indicators of probability of presence generated by the processing unit comprise each indicator of the selection as well as each indicator of probability of presence, generated for the time window, not selected.

In a second embodiment, the data processing unit is configured to implement the following steps:
When a selection among the indicators of probability of sets of indicators of probability of presence consists of the sets of indicators of probability of presence, generating, for each arrhythmia of the plurality of arrhythmias, using a set of elementary classifiers, one of the sets of indicators of probability of presence of the arrhythmia over a time window from the descriptors of a set of at least one lead of an electrocardiogram of a patient acquired over the time window, so as to generate the indicators of probability of presence of the sets of indicators of probability of presence,
Generating, among the indicators of probability of presence of the sets of indicators of probability of presence, only each indicator of probability of presence of the selection, when the selection comprises an incomplete part of the indicators of probability of presence of the sets of indicators and is not empty.

The data processing unit is configured to use for this purpose, at least one classifier of the set of elementary classifiers that uses at least one descriptor of at least one lead of the set of leads of the electrocardiogram of the patient acquired over the time window.

In other words, only each elementary classifier capable of generating, i.e. configured to generate, an indicator of the selection is used. This embodiment is therefore economical in terms of calculations.

Advantageously, in the latter embodiment, when the selection is null, no indicator is generated for the time window.

Consequently, generally speaking, the data processing unit is configured to implement the following steps:
when a selection of indicators among the sets of indicators of probability of presence consists of the indicators of probability of presence of the sets of indicators of probability of presence, generating, for each arrhythmia of the plurality of arrhythmias, using a set of elementary classifiers, one of the sets of indicators of probabilities of presence of the arrhythmia over a time window using descriptors of a set of at least one lead of an electrocardiogram of a patient acquired over the time window, so as to generate the indicators of the sets of indicators of probability of presence,
when the selection comprises an incomplete part of the indicators of probability of presence of the sets of indicators and is not empty, generate each indicator of the selection using at least one of the elementary classifiers using at least one descriptor of at least one lead of the set of at least one lead,
generating an input vector Vk, the components of the input vector Vk comprising, among the indicators of the sets of indicators of probability of presence, only each indicator of probability of presence of the selection,
generating, using a global classifier GC, global indicators of probability of presence of arrhythmias in the time window Fk from the input vector Vk,
   the system comprising a selector making it possible to select and/or deselect at least one of the indicators of probability of presence such that the selection is composed of each selected indicator of probability of presence.

The selection of indicators is made from the indicators of probability of presence of the sets of indicators of probability of presence likely to be generated for the time window, for the arrhythmias of the set of arrhythmias using, for each arrhythmia of the arrhythmia set, using the set of elementary classifiers dedicated to the arrhythmia, using descriptors of a set of at least one lead of an electrocardiogram of a patient acquired over the time window.

Indicator or indicator of probability of presence likely to be generated by an elementary classifier it taken to mean that this elementary classifier is configured to generate this indicator of probability of presence from the descriptors of a set of at least one lead of an electrocardiogram of a patient acquired over a time window.

In other words, the selection of indicators comprises only each selected indicator of probability of presence taken among the sets of indicators of probability of presence that the sets of elementary classifiers dedicated to the arrhythmias of the set of arrhythmias are configured to generate using descriptors of a set of at least one lead of an electrocardiogram of a patient acquired over the considered time window.

The selected state is, for example, the default state for each indicator. The selector INT, SE then advantageously makes it possible at least to deselect at least one of the indicators. Advantageously, but not necessarily, the selector INT, SE further makes it possible to select the indicator. This allows the indicator to be selected and deselected.

Alternatively, the deselected state is, for example, the default state of each indicator. The selector then advantageously makes it possible at least to select at least one of the indicators. Advantageously, but not necessarily, the selector INT, SE further makes it possible to deselect the indicator.

In one particular embodiment, the selector INT, SE makes it possible to select and/or deselect individually each indicator of a subset of at least one of the indicators generated or, more generally, likely to be generated by the elementary classifiers. It makes it possible, for example, to individually select the different indicators generated or, more generally, likely to be generated by the elementary classifiers.

In another embodiment, the selector makes it possible to select and/or deselect several indicators jointly.

The selector INT, SE makes it possible, for example, to select and/or deselect indicators jointly, by selecting and/or deselecting at least one elementary classifier and/or by selecting and/or deselecting at least one lead and/or by selecting and/or deselecting at least one arrhythmia and/or by selecting and/or deselecting at least one group of elementary classifiers.

For example, each elementary classifier, each lead, and each arrhythmia are capable of being in a selected state and in a deselected state.

The selected state is, for example, the default state. Alternatively, the deselected state is the default state.

According to one exemplary embodiment, the selector comprises a first selector making it possible to deselect and/or select individually the lead(s) Dkᵢ of the time window Fk such that each indicator generated or, more generally likely to be generated, from the deselected lead, by a predetermined set of elementary classifiers, is in the deselected state. This avoids taking into account the lead deselected by the global classifier GC to generate the global indicators relating to the time window Fk.

The predetermined set of elementary classifiers, for example, consists of all the elementary classifiers. Thus, the deselection of a lead results in the deselection of each indicator generated from the lead.

Alternatively, the predetermined set of elementary classifiers comprises a subset of elementary classifiers.

In the example of FIG. 3, the system comprises at least one elementary classifier, notably MO_FA, MO_FLA, PW_FLA, PW_FA configured to generate several indicators from respective leads, each indicator being generated from a single lead DK₁, Dk₂. The predetermined set comprises, for example, only these classifiers. This makes it possible to avoid the deactivation of the R-R analyzer classifier when using this lead.

Advantageously, as in the example of FIG. 3, the classifiers of the sets of classifiers formed for the different arrhythmias are classified by groups. The classifiers of a same group use the same descriptors of the same lead(s) to generate their indicators. For example, the morphological classifiers MO_FA and MO_FLA belong to a same group, just as the R-R analyzer classifiers belong to a same second group and the P wave analyzer classifiers belong to a same third group.

According to an alternative or complementary exemplary embodiment, the selector INT, SE comprises a second selector making it possible to select and/or deselect individually groups of classifiers of the sets of elementary classifiers such that each indicator generated or, more generally, likely to be generated from the deselected group is in the deselected state.

According to an alternative or complementary exemplary embodiment, the selector INT, SE comprises a third selector making it possible to select and/or deselect individually the elementary classifiers of the sets of elementary classifiers dedicated to the respective arrhythmias such that each indicator generated or, more generally, likely to be generated from each deselected classifier is in the deselected state.

According to another complementary or alternative example, the selector INT, SE comprises a fourth selector making it possible to select and/or deselect individually the arrhythmias such that each indicator generated or, more generally, likely to be generated for each deselected arrhythmia is deselected.

The different selectors may be identical or different.

Advantageously, as may be seen in FIG. 1, the processing system S comprises a user interface INT.

The user interface 120 allows a user to enter data or commands so as to be able to interact with the programs according to the invention.

The user interface INT comprises, for example, an output interface INTS and an input interface INTE.

The input interface comprises, for example, a keyboard or a pointing interface, such as a mouse, an optical pen, a touchpad, a remote control, a voice recognition device, a haptic device.

The output device INTS is designed to render information to a user, sensorily or electrically, such as, for example, visually or acoustically. The output interface comprises, for example, a graphic interface.

The output interface INTS may be the input device INTE, for example, in the case of a touch tablet.

Advantageously, the user interface INT allows a user to select and/or deselect at least one of the elementary indicators. In other words, the selector function is performed by the user interface INT.

The user interface INT allows the user to interact with the classifier C via a command module CO, which is a functional brick of the system S, for the generation of the vector V.

The output interface INTS is, for example, configured to present to the user a list of indicators likely to be selected and/or deselected and/or a list of one or more leads likely to be selected and/or deselected and/or a list of groups of individual classifiers likely to be selected and/or deselected and/or at least one list of elementary classifiers likely to be selected and/or deselected.

The input interface INTE makes it possible to receive at least one command to select or deselect the user, an indicator and/or a lead and/or a group of classifiers and/or an individual classifier taken from the list(s) displayed.

The user interface INT interacts with the vector generator GV, via the command module CO, to generate the input vector V corresponding to the command(s) of the user. The command module CO is configured to generate, from the user's selection and/or deselection commands, a command, intended for the vector generator GV so that it generates the input vector compliant with the commands of the user.

Advantageously, the user interface INT is configured to make it possible to selectively display, on a screen of the output interface INTS, at least one indicator and/or at least one global indicator and/or the sequence of states and/or at least one of the leads and/or at least one of the descriptors used by at least one elementary classifier. This helps the user define which indicators he wants to select and which he wants to deselect.

Alternatively or in addition, the selector comprises an automatic selector SE, which is a functional brick of the system S, configured to:
verify whether a condition for selecting or deselecting at least one indicator is met,
automatically select or respectively deselect said at least one indicator when the selection or respectively deselection condition is met.

For example, the automatic selector SE comprises an analyzer of characteristics of the lead(s) of the time window configured to verify whether the characteristics verify a selection or deselection condition of at least one indicator is met and a command generator, which can be performed by the command module C, configured to generate a selection or deselection command intended for the vector generator GV for it to generate the input vector V.

The selection or deselection condition is, for example, a composite condition.

For example, the characteristic analyzer is configured to verify, for each lead, whether a set of at least one quality criterion is met, comprising, for example, a first criterion according to which a signal-to-noise ratio is greater than a predetermined threshold (making it possible to verify the quality of the lead), and/or a second criterion according to which the absolute value of the mean amplitude of the lead is greater than a predetermined threshold (making it possible to verify that the signal is actually detected).

The automatic selector SE is configured to deselect each lead not complying with the quality criterion.

The automatic selector SE interacts with the vector generator SG, via the command module CO, to generate the input vector V corresponding to the user command(s). The command module CO is configured to generate, from the selection and/or deselection commands of the automatic selector SE, a command, intended for the vector generator SG so that it generates the input vector compliant with the command of the automatic selector and optionally that of the user.

The first, second and third selectors may be implemented by the same means, for example the user interface INT or the automatic selector or by separate means. As an example, one of the selectors is implemented by the user interface INT and the others by the automatic selector.

Alternatively, the system is devoid of a selector.

More generally, the different functional bricks mentioned above and likely to be artificial neural networks, may be more generally, learning functions, of which certain parameters are likely to be set by learning, during a training phase. After the training phase is completed, the parameters are set and the bricks are said to be in the trained state.

These functional bricks are, for example, in the trained state in the system S.

### Method

The invention also relates to a method for characterizing a heart rhythm. The system S and the system SYS are configured to implement the method according to the invention.

The steps of an example of the method according to the invention are shown in FIG. 5.

Advantageously, but not necessarily, the method according to the invention comprises a step of acquiring the set of at least one lead Dkᵢ over the time window Fk. This step is implemented by the acquisition system ACQ.

The method also comprises a step 15 of generating descriptors from the set of at least one lead DKᵢ.

This step is advantageously implemented using the descriptor generator GD.

Alternatively, the descriptors are generated prior to the method according to the invention. The method then begins with a step of reception of these descriptors by the system S.

Step 15 comprises a step 151 of detecting and timestamping the beats, i.e. to determine the times of occurrence or TOC of the consecutive R waves of at least one of the leads.

Step 15 of generating the descriptors also comprises the generation 160 of statistical indicators characterizing the distribution of the patient's R-R intervals during the acquisition window Fk from at least one lead of the set of at least one lead Dkᵢ.

This step is advantageously implemented by executing the statistical indicator generator GIS.

Step 160 advantageously comprises the generation 161 of an RR series, SRRk for the time window Fk, from at least one lead of the set of leads, for example the lead Dkᵢ.

This step comprises, for example, the building of an RR series by calculating the R-R intervals between consecutive beats, from the time stamps of the consecutive R waves.

It is advantageously implemented by executing the RR series generator GRR.

Step 160 advantageously comprises the generation 162, from the RR series, of at least one statistical indicator Gk characterizing the statistical distribution of the intervals R-Rk (h) of the RR series, SRRk.

This step is advantageously implemented by executing the RR series generator GRR.

This step advantageously comprises the parameterization of a Gaussian mixture model and the calculation of at least one elementary statistical indicator from at least one parameter of the parameter Gaussian mixture model.

Step 161 advantageously comprises the generation of the sequence of relationships between consecutive R-R intervals.

It then advantageously comprises, for each arrhythmia, a step 164a, 164b of generating indicators IFAk, respectively IFLAK, of probabilities of presence of the arrhythmia from the sequence of relationships between R-R intervals.

These steps are advantageously implemented by executing the RR ratio classifiers, RA_FA, RA_FLA.

Step 15 advantageously comprises a step 152 of combining, for example averaging, during which, for each lead, at least one portion of a subset of the beats present on the lead Dk₁, respectively Dk₂, and detected by the R wave detector is combined, for example averaged.

During this combination, it is possible to combine entire beats or portions of beats as explained previously.

This step is implemented by executing the combiner MO.

Step 15 advantageously comprises a portion extraction step 153, in which a portion PK₁, Pk₂ is extracted from one of the combinations or averages MK₁, Mk₂, obtained from one of the leads DK₁, Dk₂. This part comprises a portion preceding the R wave of the combination obtained.

It may correspond to the entire combination obtained or to an incomplete part of the combination obtained.

For example, this part comprises only a part of the combination or average preceding the R wave and devoid of the R wave.

This portion is advantageously likely to comprise the P wave in the case of sinus rhythm.

This portion is, for example, a part of predetermined duration preceding the R wave as explained previously.

The method then comprises a step 20 of generating indicators of probability of presence of arrhythmias of a predetermined set of arrhythmias, during the acquisition window Fk from descriptors of the set of at least one lead Dkᵢ. This step is implemented by using, i.e. by executing, the classifier C.

This step 20 comprises, the generation, during a step 21, for each arrhythmia of the plurality of arrhythmias, of a set of indicators of probabilities of presence of the arrhythmia over the time window, using a set of elementary classifiers using the descriptors. The elementary classifiers use separate respective descriptors.

In one particular embodiment, step 21 comprises the following steps, implemented for each arrhythmia:
generating, during a step 210_FA, respectively 210_FLA, a first set of indicators IMFAK₁, IMFAK₂, respectively IMFLAK₁, IMFLAK₂ of probability of presence of the arrhythmia FA; respectively FLA, in the time window Fk using a first classifier, which is a morphological classifier MO_FA, respectively MO_FLA, using values of the set of at least one lead Dk₁, DK₂,
generating, during a step 211_FA, respectively 211_FLA, a second set of indicators of probability of presence of the arrhythmia IPFA₁, IPFA₂; respectively IPFLA₁, IPFLA₂, in the time window Fk, using a second classifier, said P wave analyzer classifier PW_FA; respectively PW_FLA, using the first portions including a part preceding the R wave, of combinations of second portions of beats of the set of at least one lead DK₁, DK₂,
generating, during a step 212_FA, respectively 212_FLA, a third set of indicators IRRFAK; respectively IRRFLAK, of probabilities of presence of the arrhythmia in the time window Fk, using a third classifier known as the R-R interval analyzer classifier, RR_FA; respectively RR_FLA, from the set of statistical indicators representative of the R-R interval distribution of the electrocardiogram ECGk.

Step 212_FA, respectively 212_FLA, is advantageously implemented by executing the R-R interval analyzer classifier, RR_FA; respectively RR_FLA from a vector composed of at least one statistical indicator generated by the generator GIS, for example the indicator Gk characterizing the R-R distribution and the probability indicator generated for the arrhythmia considered IFAk, IFLAK.

In the case of several leads, at least one classifier uses both leads as described above. It then generates, during its execution, different indicators for the two leads. Advantageously, it processes the leads consecutively, so as to successively generate the indicators IMFAK₁ and IMFAK₂. This is the case for the first and second classifiers, for example.

The method then advantageously comprises a step 40 of generating an input vector Vk, by executing the input vector generator itself using the sets of indicators of probability generated during step 21 for the time window Fk.

Advantageously, the input vector Vk is generated using the selection of indicators generated in step 21 for the time window Fk.

The method then advantageously comprises a step 50 of generating global indicators IGFAK, IGFLAK, of probability of presence of arrhythmias in the time window Fk from the input vector Vk. This step is implemented by executing the global classifier GC.

Advantageously, the global indicators comprise an indicator IGRSk of probability of presence of the sinus rhythm in the time window Fk.

Advantageously, the previous steps are repeated so as to generate sequences of K sets of global indicators IGFAK, IGFLAK, IGRSk from descriptors of successive sets of at least one lead DK₁, DK₂.

In other words, the method comprises the acquisition of a sequence of sets of at least one lead, i.e. of ECGk with k=1 to K, acquired during K successive, for example consecutive, time windows F of a global acquisition window FG of global duration TG, the generation of the descriptors from the set of at least one lead acquired for each of these windows.

Alternatively, the method comprises only the reception of sets of descriptors respectively associated with sets of at least one lead acquired successively, i.e. acquired over successive time windows Fk, and implementing steps 21, 40 and 50, for each of these sets of descriptors. Each set of global indicators IGFAK, IGFLAK, IGRSk is generated from a single set of at least one lead Dk₁, Dk₂ of the time window sequence Fk. The temporal order of the sets of global indicators IGFAK, IGFLAK, IGRSk is the same as that of the sets of leads DK₁, Dk₂ from which they are respectively generated.

Advantageously, the method comprises a step 60 of time analysis, to define a sequence of states Ek of the heart rhythm corresponding to the states Ek of the heart rhythm over the successive time windows Fk from the sequence of sets of global indicators IGFAK, IGFLAK, IGRSk generated for the successive time windows Fk.

Advantageously, this step is implemented by executing the time analyzer TC from the sets of global indicators IGFAK, IGFLAK, IGRSK generated for the successive time windows.

Advantageously, the method comprises, for at least one time window Fk, a step 30 of selecting and/or deselecting at least one of the indicators generated during step 21 for the corresponding time window Fk, so that the selection of indicators is composed of each selected indicator. In step 40, the vector Vk is generated from the selection of indicators.

This step 30 is implemented by the selector.

In the example of the figure, the selection and/or deselection step is implemented after step 21.

Alternatively, the selection and/or deselection step is implemented before the step of generating indicators of probability of presence by the elementary classifiers.

Advantageously, in the alternative in which only each indicator of probability of presence of an arrhythmia of the selection is generated, a selection and/or deselection step is advantageously implemented before the step of generating the indicators.

Step 30 may be a selection step, prior to the generation of the indicators of probability of presence by the elementary classifiers, implemented once for a plurality of time windows.

Step 30 is, for example, implemented by means of the human-machine interface and/or by means of the automatic selector.

In the case of presence of the selection and/or deselection step 30, for a time window, the method may include step 40 of generating the input vector using the vector generator using each indicator in the state selected prior to the selection/deselection as well as step 50 of generating global indicators from the input vector and step 60 of time analysis from the global indicators generated for the time windows including the window Fk. The method advantageously comprises updating the input vector, the global indicators and the set of states by repeating steps 40, 50 and 60 from the selection of states obtained following the selection step 30.

The training of the training functions used in the method according to the invention is advantageously implemented prior to the method according to the invention. It is advantageously supervised, at least for the classifiers.

Advantageously, the input data of at least one classifier, for example of each of the classifiers, are pre-calibrated for use by the classifier so that they are within predetermined value ranges. The system advantageously comprises calibration means configured for these functions. And the method advantageously but not necessarily comprises these steps.

By way of example, the system SYS comprises means for calibrating the leads so that the values of the leads used by the morphological classifier are calibrated values of these leads, i.e. values within a predetermined interval. The analog converter may, for example, perform this function.

Advantageously, the training data is calibrated in the same manner.

### Hardware

From a hardware point of view, the processing system S may be seen as a calculator interacting with a computer program product.

The system S comprises at least one computer, for example, a microcomputer, a network of computers, an electronic component, a tablet, a smartphone, or a personal digital assistant (PDA).

The memory assembly MEM comprises, for example, a computer-readable medium. The computer-readable medium is a tangible device readable by the reader of the computing unit, capable of storing electronic instructions and of being coupled to communication means of the system S.

In other words, the machine-readable medium is a tangible medium. In other words, it is not a transient signal in itself, such as radio waves or other freely propagating electromagnetic waves, such as light pulses or electronic signals. Such a computer-readable storage medium is, for example, an electronic storage device, a magnetic storage device, an optical storage device, an electromagnetic storage device, a semiconductor storage device, or any combination thereof.

As an example, the readable medium is an optical disc, a magneto-optical disc, a ROM (Read-Only Memory), an EPROM (Erasable Programmable Read-Only Memory), an EEPROM (Electrically Erasable Programmable Read-Only Memory), a RAM (Random Access Memory), a magnetic card or an optical card. The memory assembly may include an operating system and load the programs according to the invention. It includes registers adapted to record parameter variables created and modified during the execution of the aforementioned programs. A computer program including software instructions is then stored on the readable medium.

The data processing unit P comprises at least one electronic circuit designed to manipulate and/or transform data represented by electronic or physical quantities in registers of the evaluation system and/or memories into other similar data corresponding to physical data in the register memories or other types of display devices, transmission devices or memory devices.

The data processing unit TR comprises, for example, memories, for storing data, operationally coupled to the data processing circuit and a drive adapted to read the computer-readable medium.

Each function of the system S is executed by causing the data processing unit TR to read a predetermined program on hardware such as the memory assembly MEM in such a way that the data processing circuit performs calculations, commands communications and reads and/or writes data to the memory assembly MEM.

The method is executed on a single computer or on a system distributed between several computers (notably via the use of cloud computing).

The data processing unit comprises at least one of the elements listed below: an assembly of one or more processors (e.g. a central processing unit (CPU), a graphic processing unit (GPU), a microcontroller and a digital signal processor (DSP) capable of interpreting instructions in the form of a computer program, a programmable logic circuit, such as an application-specific integrated circuit (ASIC), an in-situ programmable door network (FPGA), a programmable logic device (PLD) of programmable logic arrays (PLA), a system on chip (SOC), and/or an electronic board in which the steps of the method according to the invention are implemented in hardware elements.

The program product may comprise the computer-readable recording medium.

Alternatively, the program instructions are taken from an external source and downloaded via a network. This is notably the case for the applications. In this case, the computer program product comprises a computer-readable data medium on which program instructions are stored or a data medium signal on which the program instructions are encoded.

The invention relates to a computer program product comprising the computer-readable medium containing instructions which, when they are executed by the data processing circuit, cause the system S to implement the steps of the method according to the invention, i.e. to execute the functional bricks of the system according to the invention.

The form of the program instructions is, for example, a form of source code, a computer-executable form, or any form intermediate between source code and a computer-executable form, such as the form resulting from the conversion of the source code via an interpreter, assembler, compiler, link editor, or locator. Alternatively, the program instructions are a microcode, firmware instructions, status definition data, configuration data for integrated circuits (e.g. VHDL) or an object code. The program instructions are written in any combination of one or more programming languages, for example an object-oriented programming language (C++, JAVA, Python), a procedural programming language (for example language C).

The communication means allows communication between the elements of the system S and optionally between at least one element of the system and a device external to the system S. The communication means can establish a physical link between the elements of the system and/or between an element of the system and a device external to the system and/or a remote (wireless) communication link between elements of the system and/or between an element of the system and a device external to the system.

The communication means advantageously allow the transmission of data between the acquisition system ACQ and the system S. It enables for example communication between the housing and the classifier C in the associated embodiment.

1. A system for characterizing a heart rhythm comprising a data processing unit configured to generate, by computer, for each arrhythmia of a set of arrhythmias, from descriptors of a set of at least one lead of an electrocardiogram of an individual acquired during a time window, a set of indicators of probability of presence of the arrhythmia in the time window, the generation comprising:
generating a first set of indicators of probability of presence of the arrhythmia in the time window using a first classifier using values of the set of at least one lead,
generating a second set of indicators of probability of presence of the arrhythmia in the time window using a second classifier using first portions including a portion preceding an R wave, of combinations of second portions of beats of the set of at least one lead,
generating a third set of indicators of probability of presence of the arrhythmia in the time window using a third classifier using a set of statistical indicators representative of the R-R interval distribution of the electrocardiogram,
the data processing unit being configured to generate a set of global indicators comprising global indicators of probabilities of presence of arrhythmias in the time window, using a global classifier using an input vector generated from the sets of indicators generated for the arrhythmias.

2. The system according to claim 1, wherein each of the first, second, and third classifiers is configured to discriminate a sinus rhythm from the arrhythmia.

3. The system according to claim 1, wherein the data processing unit is configured to define a sequence of states of the individual's heart rhythm taken among the arrhythmias and the sinus rhythm from sets of global indicators generated for successive time windows.

4. The system according to claim 3, wherein the definition of the sequence of states uses a Viterbi algorithm configured to determine the most probable sequence of states likely to be obtained by a hidden Markov model from the sets of global indicators.

5. The system according to claim 1, wherein the first classifier comprises a convolutional neural network and/or the second classifier comprises a convolutional neural network.

6. The system according to claim 1, wherein the arrhythmias are auricular fibrillation and auricular flutter.

7. The system according to claim 1, wherein the data processing unit comprises a data processing sub-unit configured to generate the set of statistical indicators representative of the time distribution of the R-R intervals of the set of at least one lead from a sequence of R-R intervals of the electrocardiogram, the generation comprising:
parameterizing a Gaussian mixture model so as to obtain parameters of the Gaussian mixture comprising averages of Gaussians making up the model,
generating first statistical indicators of the set of statistical indicators representative of the distribution of the R-R intervals of the electrocardiogram from the Gaussian averages.

8. The system according to claim 1, wherein the data processing unit comprises a data processing sub-unit configured to generate the set of statistical indicators representative of the time distribution of the R-R intervals of the set of at least one lead from a sequence of R-R intervals of the electrocardiogram, the generation comprising the following second set of steps:
generating a sequence of relationships between consecutive R-R intervals,
generating input indicators of probabilities of presence of the arrhythmia from the sequence of relationships using an R-R ratio classifier.

9. The system according to claim 1, comprising an acquisition system (ACQ) comprising electrodes configured to acquire the set of at least one lead of the electrocardiogram of the individual.

10. The system according to claim 7, wherein the acquisition system is capable of forming an acquisition device comprising the processing sub-unit.

11. The system according to claim 1, wherein the set of at least one lead comprises several leads.

12. A method for characterizing a heart rhythm comprising a generation, implemented by computer, for each arrhythmia of a set of arrhythmias, from descriptors of the set of at least one lead of an electrocardiogram of an individual acquired during the time window, of a set of indicators of probability of presence of the arrhythmia over the time window, the generation comprising:
generating a first set of indicators of probability of presence of the arrhythmia in the time window using a first classifier using values of the set of at least one lead,
generating a second set of indicators of probability of presence of the arrhythmia in the time window, using a second classifier from first portions, preceding the R wave, of combinations of second portions of beats of the set of at least one lead,
generating a third set of indicators of probability of presence of the arrhythmia in the time window using a third classifier using a set of statistical indicators representative of the R-R interval distribution of the electrocardiogram,
generating a set of global indicators comprising global indicators of probabilities of presence of arrhythmias in the time window, using a global classifier using an input vector generated from sets of indicators generated for the arrhythmias.

13. A computer program product comprising a non-transitory readable information medium, on which a computer program comprising program instructions is stored, the computer program being loadable on a data processing unit and adapted to drive the implementation of steps of the method according to claim 12, when the computer program is implemented on the data processing unit.

14. A non-transitory readable information medium including program instructions forming a computer program, the computer program being loadable on a data processing unit and adapted to drive the implementation of steps of the method according to claim 12 when the computer program is implemented on the data processing unit.
